(19) United States
(12) Patent Application Publication
(10) Pub. No.: US 2015/0148651 A1
(43) Pub. Date: May 28, 2015

(54) Imaging System Frequency Modulation Method

(71) Applicant: Howard Letovsky, Willits, CA (US)

(72) Inventor: Howard Letovsky, Willits, CA (US)

(21) Appl. No.: 14/089,672

(22) Filed: Nov. 25, 2013

(51) Int. Cl. H03C 7/00; A61B 6/03; A61B 8/13; A61B 5/055

(52) U.S. Cl. CPC H03C 7/00; A61B 5/055; A61B 6/032; A61B 8/13
USPC 600/407; 332/117

(57) ABSTRACT

A method is disclosed herein to add therapeutic functionality to medical imaging systems. The preferred embodiment of an Imaging System Frequency Modulation Method in accordance with the present invention adds at least one bioactive frequency that is part of the acoustic or electromagnetic frequency spectrum to the imaging system output frequency used to acquire an image in a medical imaging system by modulating said imaging system output frequency with said bioactive frequency.

### FIELD OF THE INVENTION

The present invention relates to methods and apparatus used for non-invasive imaging of living tissues, organisms, and various species—including mammals. The present invention also relates to therapeutic electro-medical tools.

### BACKGROUND OF THE INVENTION

The global medical industry uses a wide variety of imaging systems. These include ultrasonic, MRI, PET, CT and XRAY scanners. For the purpose of the present invention method, any imaging system that uses any part of the acoustic or electromagnetic frequency spectrum will be considered as covered by the present invention.

For almost two hundred years, tools that use parts of the acoustic and electromagnetic spectra have been used to promote healing in damaged tissues. In general, therapeutic ultrasound and lasers work by heating tissues—essentially controlled damage of tissues—to activate collagen replacement actions and by drawing blood flow to a damaged body part to activate regenerative processes and draw the immune systems' attention to a particular area.

More recently, bioactive frequencies are being discovered and integrated into electro medical tools to directly stimulate cell regeneration symbiotically rather than by controlled damage. Alternating current frequencies in the acoustic and ultrasonic spectra are used in many types of electro medical tools available all over the world to aid in healing tissues and eliminate disease. Low frequency TENS and neuromuscular stimulators units are used to control pain and potentially enhance muscle tone. Electrical bone growth stimulators using both low acoustic frequencies radiated through cooper coils and ultrasonic frequencies radiated through metal transducers aid in the filling in and healing of gapped bones in a major fracture.

Kidney stones used to be destroyed by pulsed electrical shock waves applied to the stones through a catheter inserted into the body's urinary tract. Now, the Storz medical equipment company and others, offer a non-contact, non-invasive system that breaks up the stones at a distance ultrasonically.

Novocure is one of the first companies under FDA clinical trials in the United States to use electromagnetic frequencies to kill the reproductive functionality of brain tumors without the dangerous side effects of chemotherapy, or the adjacent tissue damage caused by radiation therapy. Novocure is in clinical trials in six countries at this time.

At the UMC Utrecht, in the Netherlands, they have constructed a prototype MRI and radiotherapy accelerator toolset combination using a modified 6 MV Elekta (Crawley, UK) accelerator operating next to, and in concert with, a modified 1.5 T Philips Achieva (Best, The Netherlands) MRI system. The side by side 1.5 T MRI system and radiotherapy accelerator system allows simultaneous irradiation and magnetic resonance imaging. No degradation of the performance of either system was found to occur. However, these are two discreet systems being used in tandem.

Tools like Cyberknife use a robot controlled radiation beam aimed at tumors with about 1 millimeter precision aided by a separate imaging tool. However, Cyberknife works by tissue ablation or destruction and has no imaging function that modulates its beam—or vice versa.

The present invention defines a new state of the art in electro-medical therapeutic imaging tools. Prior to the present invention, no electromagnetic therapy or imaging system has been constructed or modified to modulate the imaging frequency used to collect an image with another frequency designed to stimulate the healing of tissues. The present invention uses the imaging frequency waveform of a medical imaging system as a carrier wave to apply at least one bioactive frequency waveform to an organism being imaged by modulating said imaging waveform frequency with said bioactive frequency.

### SUMMARY OF THE INVENTION

The primary objective of the present invention is to modulate the imaging frequency used in a medical imaging system with at least one bioactive frequency to add tissue regeneration, stem cell activation, tumor reduction or elimination, kidney or gall stone elimination, or other beneficial effects, to the medical imaging functions of the imaging system.

To date, therapeutic electro-medical tools have been entirely separate from electromagnetic imaging systems. The present invention may combine the functions and effectiveness of the two discreet technologies by incorporating frequency generators, modulating and demodulating circuitry, amplifiers, microprocessors, other electrical hardware, or software to existing or yet to be produced medical imaging systems.

In electronics and telecommunications, modulation is the process of varying one or more properties of a periodic waveform, called the carrier Frequency, with a modulating frequency, which typically contains information (or energy) to be transmitted.

A device that performs modulation is known as a modulator and a device that performs the inverse operation of modulation is known as a demodulator.

In the case of the present invention, the imaging frequency may be modulated with at least one additional bioactive frequency and the combined modulated frequency signal may be demodulated in two ways. It may be demodulated electronically on the image processing side of the imaging system to preserve the imaging functionality of the system, and a living organism is intended to concurrently demodulate the modulated transmitted imaging waveform as it radiates through said organism.

All living tissues can be affected by certain acoustic and electromagnetic waves, but the specific effects vary dramatically in relation to the power and frequency of the Frequency being applied—as well as the tissue type, density, and depth within an organism that the tissue is located. It is therefore desired to employ modulated bioactive frequencies that may interact with living tissues.

A more complete understanding of the present invention, as well as further features and advantages, will be obtained by reference to the following detailed description and drawings. Preferred embodiments of the present invention will be described in the claims.

### BRIEF DESCRIPTION OF THE DRAWINGS

FIG. 1 is a schematic component diagram illustrating an Imaging System Frequency Modulation Method per the present invention.

### DETAILED DESCRIPTION OF THE INVENTION

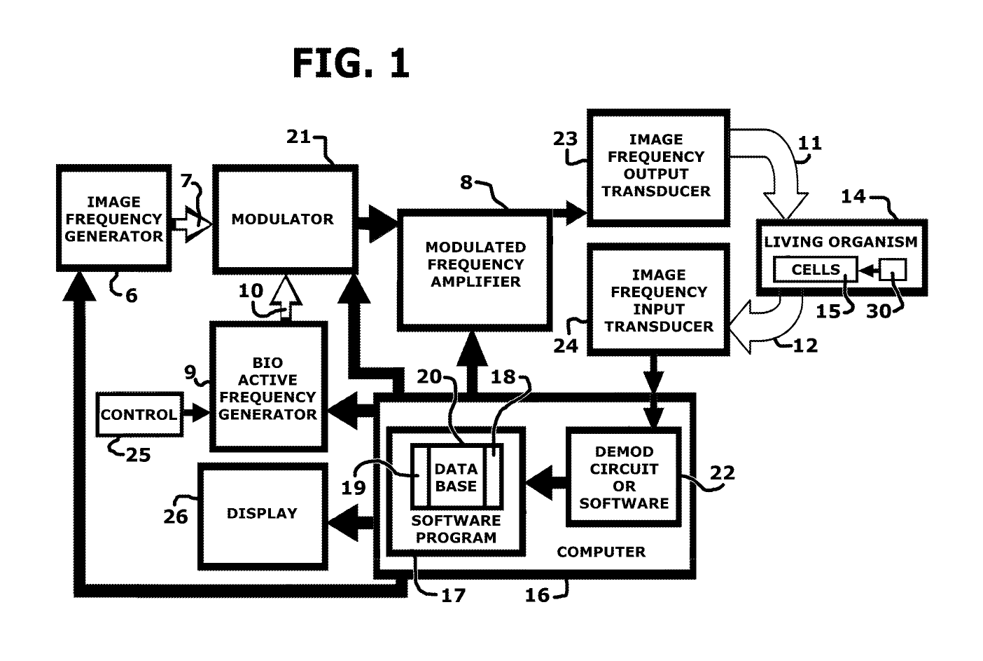

The preferred embodiment of an Imaging System Frequency Modulation Method in accordance with the present invention per the system flow chart schematic as shown in FIG. 1, adds at least one bioactive frequency 10 that is part of the acoustic or electromagnetic frequency spectrum to the imaging system output frequency 7 used to acquire an image in a medical imaging system, by combining said imaging system output frequency 7 with said bioactive frequency 10 in modulator 21.

As presented in FIG. 1, the present invention method may be applied to any imaging system, which may be an X-Ray, MRI, CT, Ultrasound, or any other imaging system designed to provide images of tissues or cell(s) 15 on or in a living organism 14. The primary variations in any imaging system are the imaging system output frequency 7 used and the type of imaging system input transducer 24 or the type of imaging system output transducer 23 used to transmit and receive the imaging frequencies. For example, in MRI and CT scanners, the transducers are a combination of large coils and magnets. In ultrasound imagers the input and output transducers are combined physically as metal beam formers. The present invention method applies to any imaging system that uses at least one frequency generator, digital to analog converters, analog to digital converters, amplifiers, image processors, computers, video displays, and at least one transducer to transmit or receive an imaging system output frequency 7.

The modulation, demodulation, frequency generator, amplifier, transducer, computer hardware, and software elements referred to in the present invention are used in various industries and disciplines so they are not detailed herein beyond the functional description presented. However, the present invention is a unique and novel method and system disclosure incorporating functions, features, and component combinations not found in any other prior art method.

The process of modulation in telecommunications and the present invention method uses an information frequency (bioactive frequency 10) to vary some parameter of a carrier (imaging system output frequency 7) frequency. The imaging system output frequency 7 may be a fixed or variable frequency—including sine, square, or other direct current, alternating current, or pulsed waveform. In most medical imaging systems, the imaging system output frequency 7 is typically a fixed frequency—ideally lending itself to be used as a carrier frequency per the present invention. The carrier waveform—or imaging system output frequency 7 waveform—in the present invention—has amplitude, frequency or phase, which can be modulated (varied) with a bioactive frequency 10 through a modulator 21 to apply modulated imaging output frequency 11 to a living organism.

Modulating an imaging system output frequency 7 waveform means requires varying one of the parameters of an imaging system output frequency 7 waveform: amplitude, frequency or phase. Therefore, the types of modulation may be categorized as:

a) AM (amplitude modulation), which can be achieved by passing the imaging system output frequency 7 through modulator 21, which be hardware or software, and may also control an amplifier 8 whose gain depends on the frequency to be encoded. This applies an envelope to the amplitude of the imaging waveform. Amplifier 8 may be part of the imaging system output circuitry as well.
b) FM (frequency modulation or phase modulation) wherein the frequency of the imaging system output frequency 7 is varied to transmit the bioactive frequency 10. The characteristics of phase modulation are very similar to FM and so the terms are often used interchangeably—since the frequency modulation of a carrier frequency is the rate of change of its phase.
c) PM (Pulse Modulation) is simply turning the imaging system output frequency 7 on or off at a particular rate.

The present invention method may use the above-described techniques or any other available analog or digital modulation technique in modulator 21. The imaging frequency modulation method may include the following or other additional aspects: wide frequency range, multiple frequency outputs, frequency sweeps, adjustable duty cycle, pulse function, sine/square/other waveform output, variable power level, visual display, and programmability.

At the receiving end of the modulated frequency transmission, a demodulator 22 reverses the modulation process. In the context of the present invention, demodulation will be effected in an imaging system by demodulator 22 which may be configured as a hardware circuit, or in the image processing section of software program 17 with demodulator 22 as an algorithm in software program 17 residing on computer 16 to preserve the living organism 14 image integrity.

Since modulating an imaging system output frequency 7 may induce errors in the image data derived from said modulated imaging output frequency 11 being applied to a living organism 14 through imaging system output transducer 23, any image frequency errors must be corrected for, or “demodulated” to provide accurate imaging capability. This is done by passing the received modulated imaging frequency 12—as received by imaging system input transducer 24 through demodulator 22, which may be electrically coupled to, or within computer 16 or software program 17—whether it is operating in the analog domain, or through a digital demodulator if it is in digital form—or through an analog to digital converter then through a digital demodulator in the image processing stage, during which software program 17 applies demodulator 22 to the received modulated imaging frequency 12. Demodulator 22 may remove any bioactive frequency 10 from the received modulated imaging frequency 12 prior to storing or displaying any living organism 14 or cell 15 image data.

When a modulated imaging output frequency 11 is applied to a living organism 14, the living organism 14 itself may also demodulate the modulated imaging output frequency 11 as it passes through said living organism 14—but only a modulated imaging output frequency 11 carrying a bioactive frequency 10 that matches the demodulation capabilities of the living organism 14 may be effective. Essentially, living organism 14 frequency demodulation may consist of the absorbance, reflectance, and transmission (pass through) characteristics a given living organism 14 or cell 15 or group of cell(s) 15 in response to any bioactive frequency 10 carried by the modulated imaging output frequency 11 waveform.

The preferred embodiment of an Imaging System Frequency Modulation Method in accordance with the present invention per the system flow chart schematic as shown in FIG. 1 is intended to provide the capability to collect images of, and simultaneously affect a specific live cell 15 or cell(s) 15 in a living organism 14. The present invention is intended to observe and collect data on said effects, and use said data to optimize said effects to stimulate a specific cell 15 cellular behavior alteration 19.

As presented in FIG. 1, a bioactive frequency generator 9 may be electrically coupled to an imaging frequency generator 6 and an imaging system amplifier 8 through modulator 21 to modulate an imaging system output frequency 7 transmitted by imaging system output transducer 23 with a bioactive frequency 10 waveform to affect a cell 15 in a living organism 14 being viewed by an imaging system.

Bioactive frequency generator 9 may output a bioactive frequency 10 including but not limited to one or more waveforms configured as sine, square, ramp, pulse, noise, DC, as well as any user defined arbitrary bioactive frequency 10 with or without sweep functionality, variable duty cycle, variable phase, variable frequency, or variable amplitude within the acoustic and electromagnetic frequency spectra. For the purposes of the present invention said bioactive frequency generator 9 frequency range and number of different simultaneous frequencies that can be applied may only be limited by the current state of the art.

Bioactive frequency generator 9 may also be connected to computer 16 to allow data transfer and functional control by software program 17 residing on said computer 16. All components presented herein and added to an imaging system may be connected to computer 16 for bidirectional data transfer. The data couplings between computer 16 and all other components described in the present invention may be USB, RS27, firewire, GPIB, Thunderbolt, or any other industry standard instrumentation data interface. The electrical wiring between all components may be BNC cables or any other industry standard, and as such are not necessary to detail or number herein. Said data couplings are represented in FIG. 1 by thick black shaft lines with black arrowheads. Imaging system output frequency 7 and bioactive frequency 10 are represented with white shaft and arrowheads placed inside the black data couplings.

Amplifier 8 may have frequency response equal to bioactive frequency generator 9. Bioactive frequency generator 9 may also have a manual control input 25 that is electrically coupled to computer 16 to allow a user to choose an output frequency of bioactive frequency generator 9.

A video display 26 may be coupled to computer 16 to allow a user to monitor any effect of any modulated imaging output frequency 11 on any cell 15 in a living organism 14. Software program 17 may be configured with a database 20 that may include a cell 15 baseline cell behavior information lookup table 18.

Lookup table 18 may include typical cell size for a given cell type, rate of mitosis, molecular pathway openings and closings relative to certain chemical compounds, etc. Said software program 17 may also be configured to track and map any cell 15 in living organism 14 and populate said database 20 with cellular behavior alteration data 19 derived received by imaging system input transducer 24, and compare said cellular behavior alteration data 19 with said lookup table 18. Cellular behavior alteration 19 may include any cell 15 behavior deviations from lookup table 18, including such changes as size and shape. As lookup table 18 and database 20 are refreshed and updated in response to cellular behavior alteration 19, software program 17 may be configured to modify any bioactive frequency 10 in response to any cellular behavior alteration 19 occurring in response to any modulated imaging output frequency 11.

Software program 17 may be configured to track a plurality of cell(s) 15 in a living organism 14 simultaneously. In database 20, a cell 15 or cell(s) 15 location within living organism 14 may be represented in the x/y/z axes relative to a “zero” point on a three dimensional environment model mapped to the observable area of an imaging system in database 20 at a resolution only limited by current state of the art in medical imaging technology. This type of “object of interest” targeting and tracking software is now available from an array of imaging system providers.

Any cellular behavior alteration 19 data may be logged and updated in real time—continually updating lookup table 18 in database 20 in software program 17. Statistical outputs from cellular behavior alteration 19 data may include:

a. real time updated position information of any cell 15 in a living organism 14.
b. acceleration/deceleration of any cell 15 in real time and over time.
c. expansion/contraction of any cell 15 in real time and over time.
d. ambient fluid flow into/out of any cell 15 in real time and over time.

A user of the present invention, through control input 25, may initially set bioactive frequency generator 9 to sweep through the frequency limits of said bioactive frequency generator 9 at a particular sweep rate not to exceed the imaging and data acquisition limits of an imaging system. Control input 25 may be a knob, joystick, or any other standard industry means to vary the output of bioactive frequency generator 9. When any cellular behavior alteration 19 occurs, a bioactive frequency 10 being output at that moment may be locked in by a user through control input 25, or by algorithms within software program 17, and said bioactive frequency 10 amplitude may be raised or lowered or the pulse width or duty cycle may be altered. Bioactive frequency generator 9 may then be directed by a user or software program 17 to add one or more additional bioactive frequency 10 waveforms, as well as additional harmonic frequencies of said bioactive frequency 10, as well as wave shape and amplitude until no further cellular behavior alteration 19 occurs.

A user may manually choose to lock-in or sweep through any bioactive frequency 10 that is output by bioactive frequency generator 9. Software program 17 may also be configured to sweep through or lock in any bioactive frequency 10 of bioactive frequency generator 9.

Any bioactive frequency 10 that is most absorbed by a cell 15 may be considered for the purposes of the present invention as a resonant frequency of a cell 15. Any said resonant frequency may be identified through laboratory experimentation, and then may be integrated within database 20 as part of lookup table 18. “Overdriving” the amplitude of a resonant frequency with respect to a base rate of a bioactive frequency 10 absorption of a cell 15, may affect the electrical conductivity and the chemical and mechanical behavior of a cell 15. A bioactive frequency 10 may then be manipulated and augmented by changes in frequency, pulse rate, amplitude, and wave shape, as well as the addition of additional frequencies, frequency inversions, harmonics, or related dissonant frequencies of any said bioactive frequency 10 by software program 17 through computer 16 and bioactive frequency generator 9. It may be the variations of a bioactive frequency 10 that catalyze a cellular behavior alteration 19.

A bioactive frequency 10 may be applied to catalyze destruction of a cancerous cell 15—initially by altering a single specific structure or behavior within a cell 15, and then outputting or altering an additional applied bioactive frequency 10 or a harmonic of said bioactive frequency 10, which may then propagate behavior changes in other cell 15 structures and mechanics like a domino effect—possibly allowing an immune system to recognize a cancer cell 15 within a living organism 14 as an invader and dispatch white blood cells to destroy it. For example, if a molecule of a given cell 15 may be comprised of ten atomic elements arranged in a particular way, modifying the polarity of the third most abundant atomic element in said molecule may have such a catalyzing effect on a cell 15 mechanics.

A bioactive frequency 10 may be applied to a cell 15 through imaging system output transducer 23 to stimulate a cellular behavior alteration 19. Said cellular behavior alteration 19 may be mapped by software program 17 in response to image data derived from imaging system 35.

A catalytic bioavailable media 30 which may include a drug, a vitamin, or a mineral compound, may also be applied to a living organism 14 in conjunction with a bioactive frequency 10, to stimulate a cellular behavior alteration 19. Said catalytic bioavailable media 30 is represented in FIG. 1 as a box with an arrowhead aimed at a cell 15 within living organism 14.

It is to be understood that the embodiments and variations shown and described herein are merely illustrative of the principles of this invention and that various modifications may be implemented by those skilled in the art without departing from the scope and spirit of the invention.

I claim:

1. An Imaging System Frequency Modulation method comprising; electrically coupling at least one waveform generator to the imaging frequency generation circuitry of an imaging device configured to capture an image of an organism, said at least one waveform generator configured to vary at least one characteristic of said imaging frequency generated by said imaging device.

2. An Imaging System Frequency Modulation method according to claim 1 that includes; electrically coupling at least one manual or automatic control device to said at least one waveform generator or said imaging device, wherein an operator or a microprocessor may vary said at least one characteristic of said imaging frequency generated by said at least one waveform generator or said imaging device.

3. An Imaging System Frequency Modulation method according to claim 1 that includes; integrating a demodulation function into an imaging system to maintain the imaging capability of said imaging system following any variation of any said at least one characteristic of said imaging frequency generated by said at least one waveform generator or said imaging device.

4. An Imaging System Frequency Modulation method according to claim 1 that includes: using frequencies from the acoustic and electromagnetic spectra in said at least one waveform generator or said imaging device.

5. An Imaging System Frequency Modulation method according to claim 1 wherein varying said at least one characteristic has an effect said organism being imaged by said imaging device.

6. An Imaging System Frequency Modulation method according to claim 1 that includes creating an electrically conductive path in said organism.

7. An Imaging System Frequency Modulation method according to claim 1 that includes speeding up the rate of mitosis in said organism.

8. An Imaging System Frequency Modulation method according to claim 1 that includes causing adult differentiated cells in said organism to exhibit stem cell behavior.

9. An Imaging System Frequency Modulation method according to claim 1 that includes interrupting or destroying the mitosis of said organism.

10. An Imaging System Frequency Modulation method according to claim 1 that includes changing the behavior of any cancerous cells in said organism so that said cancerous cells are recognized by the immune system of an organism containing said organism.

11. An Imaging System Frequency Modulation method according to claim 1 that includes causing any cell in said organism to absorb or reject a chemical compound.

12. An Imaging System Frequency Modulation method according to claim 1 that includes using at least one software database program, said at least one database including live cell sample information, and acoustic and electromagnetic frequency spectrum information.

13. An Imaging System Frequency Modulation method that includes using an imaging system; at least one computer; at least one waveform generator capable of providing at least one frequency from the acoustic or electromagnetic spectra; said at least one computer able to control said at least one waveform generator; said at least one waveform generator coupled to said imaging system to apply said at least one frequency to a living organism; wherein said at least one computer may be directed to control the parameters of said at least one frequency to cause a change in the behavior of said living organism.
